(19) United States
(12) Patent Application Publication
(10) Pub. No.: US 2015/0313774 A1
(43) Pub. Date: Nov. 5, 2015

(54) METHOD OF MAKING AN ELASTIC LAMINATE

(71) Applicants: Dieter HOMOELLE, Ochtrup (DE); Marcus Schoenbeck, Versmold (DE)

(72) Inventors: Dieter HOMOELLE, Ochtrup (DE); Marcus Schoenbeck, Versmold (DE)

(21) Appl. No.: 14/267,313

(22) Filed: May 1, 2014

(51) Int. Cl. A61F 13/56

(52) U.S. Cl. CPC A61F 13/5622

(57) ABSTRACT

A method of making an elastic laminate has the steps of first stretching a longitudinally elongated elastic film transversely and then relaxing the transversely stretched film. The relaxed and transversely stretched film is then cut into a pair of adjacent longitudinally extending strips that are bonded adjacent each other between two unstretched and longitudinally extending nonwoven webs to form a laminate. This laminate is then transversely stretched at least at the strips and then relaxed and wound up into a roll. Closure elements that have an elastic center region and less elastic end sections at both ends can be punched from the laminate thus made.

### FIELD OF THE INVENTION

The present invention relates to a method of making an elastic laminate. More particularly this invention concerns it concerns a laminate for use in a fastener of a disposable diaper.

### BACKGROUND OF THE INVENTION

The invention is directed to a method in which elastic strips are laminated next to one another between nonwoven material webs. The nonwoven material webs are supplied without prestretching and joined to the strips. The laminate thus formed is then stretched transverse to the web direction in regions rendered elastic by the laminated strips, and after elastic relaxation is wound into a roll. Due to the stretching of the laminate, which is also referred to as mechanical activation, the elastic properties of the laminate transverse to the web direction of the material web (CD direction) are improved.

A method having the described features is known from U.S. Pat. No. 7,470,340 for example. Elastic elements for hygiene products, in particular elastic closure strips for diapers, may be punched from the laminate, the elastic elements each having an elastic center region and less elastic sections at both ends. The inelastic or less elastic end regions are used to fasten hook-and-loop elements, for example hook tapes, and to attach the elastic element to inelastic surfaces of a diaper. The laminate is manufactured as a wide web having a plurality of laminated elastic strips. Closure strips necessary for diaper manufacture may be punched from the resulting multiuse material.

A method of making an elastic nonwoven laminate is known from US 2007/0237924 and 2012/0018083 in which an elastic film is preactivated by stretching, followed by elastic relaxation, then stretched again, and in the stretched state is laminated onto a nonwoven web. The stretching forces to which the elastic film is subjected during the lamination onto the nonwoven web may be reduced by preactivating the elastic film. The bond strength between the layers of the laminate may also be improved by preactivating the film. However, the lamination procedure using an elastic film that is stretched during the lamination process is complicated and not technically practical when a number of elastic strips oriented parallel to one another must be laminated between two nonwoven material webs, and a laminate having elastic and inelastic regions is to be made.

In a method known from US 2006/0003656 for making an elastic nonwoven laminate, an elastic film is stretched transverse to the web direction, and after elastic relaxation is laminated onto a nonwoven web that is stretched in the web direction and in the stretched state is bonded to the elastic film. As the result of stretching the nonwoven web, the material width of the nonwoven is reduced, and folds are formed in the nonwoven web that impart transverse elasticity to the laminate (CD direction). However, it is not possible to use the described method to make a laminate that has adjacent elastic and inelastic sections in the web longitudinal direction.

### OBJECTS OF THE INVENTION

It is therefore an object of the present invention to provide an improved method of making an elastic laminate.

Another object is the provision of such an improved method of making an elastic laminate that overcomes the above-given disadvantages, in particular that has nonwoven cover layers and elastic strips between the cover layers, and that may be stretched with a low force over a large stretching area only in the areas in which the strips are laminated.

### SUMMARY OF THE INVENTION

A method of making an elastic laminate has according to the invention the steps of first stretching a longitudinally elongated elastic film transversely and then relaxing the transversely stretched film. The relaxed and transversely stretched film is then cut into a pair of adjacent longitudinally extending strips that are bonded adjacent each other between two unstretched and longitudinally extending nonwoven webs to form a laminate. This laminate is then transversely stretched at least at the strips and then relaxed and wound up into a roll. Closure elements that have an elastic center region and less elastic end sections at both ends can be punched from the laminate thus made.

Stretching the elastic film mechanically preactivates a component of the laminate and results in an improvement in the stretching behavior of the laminate. The preactivation of the elastic film has a positive effect on the stretching force profile of the laminate and contributes to the laminate being easily stretchable over a large stretching area while greatly increasing the stretching resistance for a yield strength determined by the preactivation of the elastic film, the stretching resistance being readily determined as the yield strength upon subsequent use of the laminate. In addition, the elastic relaxation behavior of the laminate after strain relief may be improved by using a preactivated elastic film. However, the preactivation of the elastic film does not replace the mechanical activation of the laminate, but instead cooperates with it synergistically. In the preactivation of the elastic film, the film is preferably stretched essentially uniformly over its entire width. In contrast, the stretching of the laminate for mechanical activation is locally limited to the regions of the laminate that are already elastic due to the laminated strips that are preactivated according to the invention. Due to the stretching of the laminate, fibers of the nonwoven layers are irreversibly stretched in the elastic regions of the laminate, and bonding of the nonwoven in the elastic regions is reduced due to fiber tears and fiber rearrangements. This is accompanied by a renewed mechanical effect on the material of the elastic strips, as well as a mechanical effect on localized bonds between the film surface and adjacent fibers. Areas of the laminate between the elastic regions are not altered by stretching the laminate and retain the properties of the nonwoven.

For purposes of the preactivation, the elastic film is preferably stretched transversely by 100% to 500%. These numerical values refer to the change in length of the film transverse to the web longitudinal direction relative to the starting width of the film. The value of 100% means that the film in the stretched state has a width that is twice the starting width of the film. The stretching is not fully reversible. As the result of inelastic portions of the film, after its elastic relaxation the film has a slightly greater width than prior to the stretching. The width subsequent to the elastic relaxation may be approximately 10% to 30% greater than the starting width of the elastic film prior to transverse stretching.

For preactivation of the elastic film, i.e. for the transverse stretching of the elastic film prior to its further processing, a stretching roller system composed of intermeshing profile rollers is preferably used. The profile rollers may in particular be composed of multiple disks that are combined into sets, the disks preferably being arranged equidistantly for uniform stretching transversely of the web.

After preactivation, the elastic film is cut into strips. The strips are guided over deflectors and may be supplied as parallel strips to a lamination unit where the strips are laminated between nonwoven webs that are supplied on the top and bottom faces. The elastic strips are advantageously spaced from one another. The spacing between the strips may be set by positioning the deflectors. The nonwoven webs are directly joined together in the gaps between the elastic strips. It is also within the scope of the invention that the areas between the elastic strips are reinforced by colaminated reinforcing strips. Elastic and inelastic regions may thus be formed in the laminate.

For the mechanical activation, the laminate may be guided through a nip between two profile rollers, each including at least two disk sets having a plurality of disks, situated on an axis. The laminate is stretched in places by intermeshing disk sets of the two profile rollers. In roller sections between the disk sets, the profile rollers form a gap, through which the laminate is guided essentially without transverse stretching.

Relative to the overall width of the laminated strips, the maximum transverse stretching of the laminate for the mechanical activation corresponds to the value by which the elastic film is stretched for purposes of preactivation. In other words, in the area of the laminated strips, the maximum stretching of the laminate is as great as that of the elastic film during its preactivation. The transverse stretching of the laminate for the mechanical activation (relative to the overall width of the laminated strips) is preferably 50% to 90% of the value by which the film is stretched for purposes of preactivation.

A film composed of a polyolefin elastomer is preferably used as elastic film. The preactivation of the elastic film is particularly effective when an elastic film based on polyolefin elastomers is used.

A single-layer film or a multilayer film having an elastomeric core layer composed of styrene-isoprene-styrene (SIS) block copolymers, styrene-butadiene-styrene (SBS) block copolymers, styrene-ethylene/butylene-styrene (SEBS) block copolymers, polyurethanes, ethylene copolymers, or polyether block amides may also be used as elastic film.

The nonwoven from which the cover layers of the laminate are made has fibers made of stretchable polymers that have only slight elasticity compared to the polymer of the elastic film. The nonwoven may be composed of melt-blown fibers, staple fibers, or continuous fibers, the fibrous web formed from the fibers being mechanically, thermally, or chemically bonded. In particular, spunlace nonwovens may also be used as cover layers.

### BRIEF DESCRIPTION OF THE DRAWING

The above and other objects, features, and advantages will become more readily apparent from the following description, reference being made to the accompanying drawing in which:

FIG. 1 is a schematic top view illustrating the method and apparatus of this invention; and

FIG. 2 is a vertical section taken along line II-II through the transverse stretcher.

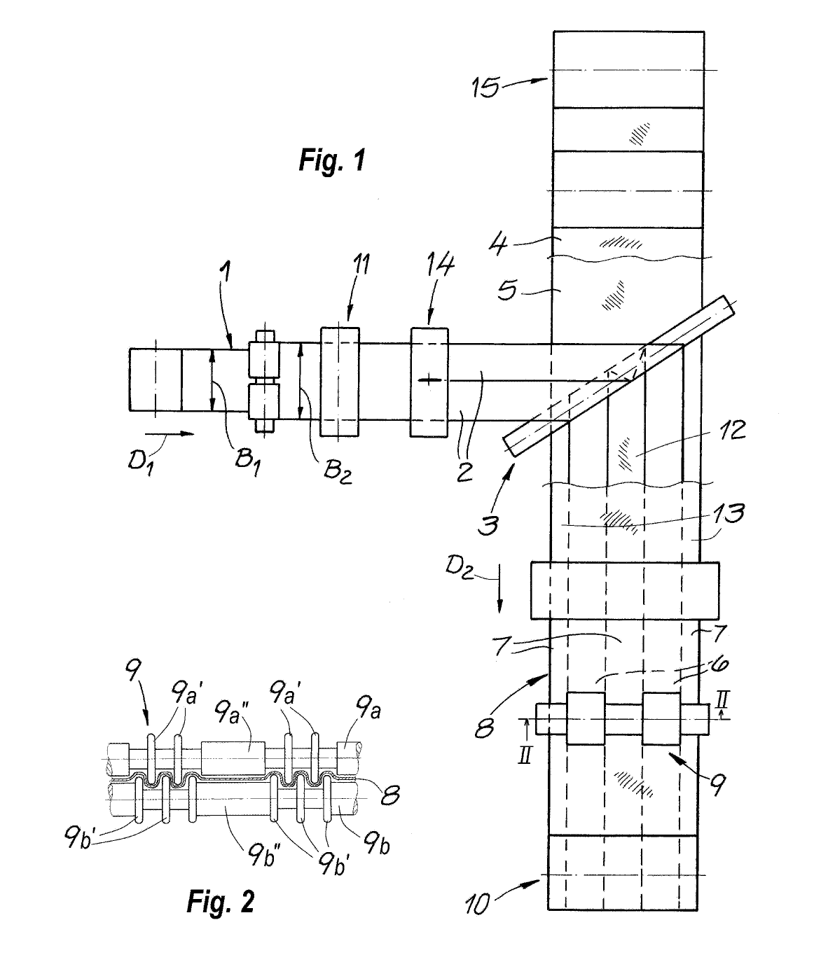

### SPECIFIC DESCRIPTION OF THE INVENTION

As seen in the drawing, an elastic film 1 is stretched transverse to a first web travel direction D₁ in a preactivation station 11, and after elastic relaxation is formed at a cutting station 14 into two parallel strips 2. The strips 2 are guided over deflectors 3, and as parallel strips are deflected through 90° to a perpendicular travel direction D₂ and laminated next to one another between two nonwoven material webs 4 and 5 fed in from a supply 15. The material webs 4 and 5 are guided above and beneath the strips 2 without prestretching, and are adhesively or thermally bonded to the strips 2. The view clearly shows that the elastic strips 2 are laminated at a gap-forming transverse spacing from one another between the cover layers formed by the webs 4 and 5, and that these nonwoven cover layers 4 and 5 are directly joined together in the gap 12 between the elastic strips 2 and at edge strips 13. Elastic regions 6 and inelastic regions 7 are thus formed in the laminate 8. The laminate 8 is supplied to an activation unit 9 in which it is stretched transverse to the web direction D₂ in the regions 6 rendered elastic by the laminated strips 2. After elastic relaxation, the laminate 8 is wound into a roll 10.

The elastic film 1 is here stretched transverse to the web direction by more than 50% in the preactivation station 11. The stretching occurs essentially uniformly over the entire width of the film 1. The elastic film is preferably stretched by 100% to 300% relative to a starting width B₁ of the elastic film, stretching to 500% also being possible. After the elastic relaxation, the elastic film 1 has a width B₂ that is 10% to 30% greater than the starting width B₁. The stretching of the elastic film 1 constitutes a preactivation that has an advantageous effect on the elongation values of the laminate 8. A stretching roller system composed of intermeshing profile rollers may be used to preactivate the elastic film 1.

A single-layer elastomer film or a multilayer film having an elastomeric core layer composed of styrene-isoprene-styrene block copolymers, styrene-butadiene-styrene block copolymers, styrene-ethylene/butylene-styrene block copolymers, polyurethanes, ethylene copolymers, or polyether block amides may be used as elastic film. An elastic blown film composed of a polyolefin elastomer is particularly preferably used.

The stretching of the laminate 8 is limited to the regions of the laminate 8 that have been made elastic by the laminated and preactivated strips 2. For this purpose as shown in FIG. 2, the laminate 8 is guided through a nip between two profile rollers 9a and 9b that include at least two sets of a plurality of disks 9a′ and 9b′ mounted on an axle. The laminate is stretched in places by the intermeshing disks 9a′ and 9b′ of the two profile rollers 9a and 9b. As a result of the stretching, textile structures of the cover layers are altered in the elastic regions 6 of the laminate, and the elongation properties of the laminate 8 in the transverse direction, are improved. Relative to the overall width of the laminated strips 2, the maximum transverse stretching of the laminate during stretching corresponds to the value by which the elastic film 1 is stretched during preactivation. The transverse stretching of the laminate 8 relative to the overall width of the laminated strips is preferably 50% to 90% of the value by which the elastic film 1 is stretched during preactivation. Between the sets of disks 9a′ and 9b′, the rollers 9a and 9b have plain cylindrical roller sections 9a″ and 9b″ in which the laminate is not subjected to transverse stretching. These regions 9a″ and 9b″ define a nip through which the laminate 8 is guided essentially without transverse stretching.

We claim:

1. A method of making an elastic laminate, the method comprising the steps of sequentially:
a) stretching a longitudinally elongated elastic film transversely;
b) relaxing the transversely stretched film;
c) cutting the relaxed and transversely stretched film into a pair of adjacent longitudinally extending strips;
d) bonding the strips adjacent each other between two unstretched and longitudinally extending nonwoven webs to form a to laminate;
e) transversely stretching the laminate at least at the strips;
f) relaxing the transversely stretched laminate; and
g) winding the relaxed and transversely stretched laminate up into a roll.

2. The method defined in claim 1, wherein during step a) the elastic film is stretched essentially uniformly over its entire transverse width.

3. The method defined in claim 2 wherein in step a) the elastic film is stretched transversely by 100% to 500% of its transverse width.

4. The method defined in claim 1, wherein step a) is carried out by passing the web between a pair of intermeshing profile rollers.

5. The method defined in claim 1, wherein in step d) the strips are bonded between the nonwoven at a transverse spacing from each other, the method further comprising the step of:
bonding together the nonwovens in a longitudinally extending gap between the strips and at longitudinally extending edge regions flanking the strips so as to form inelastic regions where the nonwovens are bonded together and elastic region at the strips.

6. The method defined in claim 1, further comprising the step between steps c) and d) of:
b′) passing the strips over a deflector so as to separate the strips transversely from each other.

7. The method defined in claim 1, wherein step e) is carried out by passing the web between a pair of profile rollers having at the strips respective sets of intermeshing disks and between the sets plain regions that do not stretch the nonwoven webs.

8. The method defined in claim 1, wherein the amount of transverse stretching of step e) is at most equal to that of step a).

9. The method defined in claim 8, wherein the stretching of step e) is equal to between 50% and 90% of the stretching of step a).

10. The method defined in claim 1, wherein the elastic film is a polyolefin elastomer.

11. The method defined in claim 1, wherein the elastic film is multilayer film having an elastomeric core layer composed of styrene-isoprene-styrene block copolymers, styrene-butadiene-styrene block copolymers, styrene-ethylene/butylene-styrene block copolymers, polyurethanes, ethylene copolymers, or polyether block amides.
